(19) United States
(12) Patent Application Publication
(10) Pub. No.: US 2020/0368045 A1
(43) Pub. Date: Nov. 26, 2020

(54) PROSTHETIC LEG APPARATUSES AND METHODS OF USING SAME

(71) Applicant: Copli Technologies Ltd., Shoham (IL)

(72) Inventors: Oz BENDERMAN, Kfar-Haim (IL); Chen BARAK, Shoham (IL)

(73) Assignee: Copli Technologies Ltd., Shoham (IL)

(21) Appl. No.: 16/635,194

(22) PCT Filed: Jul. 31, 2018
(86) PCT No.: PCT/IL2018/050850

§ 371 Date: Jan. 30, 2020

Related U.S. Application Data
(60) Provisional application No. 62/538,864, filed on Jul. 31, 2017.

(51) Int. Cl. A61F 2/80

(52) U.S. Cl. CPC A61F 2/80; A61F 2002/6614

(57) ABSTRACT

A prosthetic leg apparatus for use with a user's stump, comprising: a socket, wherein the socket is constructed of a thermoplastic material; a pylon assembly, connected to the socket and adjustable in length; and, a foot portion, connected to the pylon assembly.

### RELATED APPLICATION

This application claims the benefit of priority under Article 8 PCT of U.S. Provisional Patent Application No. 62/538,864 filed Jul. 31, 2017, the contents of which is incorporated by reference herein in its entirety.

### FIELD AND BACKGROUND OF THE INVENTION

The present invention, in some embodiments thereof, relates to the health care field and, more particularly, but not exclusively, to prosthetic limbs.

### SUMMARY OF THE INVENTION

There is provided in accordance with an aspect of the invention, a prosthetic leg apparatus for use with a user's stump, comprising: a socket, wherein the socket is constructed of a thermoplastic material; a pylon assembly, reversibly connected to the socket and adjustable in length; and, a foot portion, reversibly connected to the pylon assembly.

In an embodiment of the invention, the apparatus further comprises at least one strap attached to the socket for securing the socket to the user's stump.

In an embodiment of the invention, the at least one strap is attached to the socket at least one slot disposed on the socket.

In an embodiment of the invention, the pylon assembly includes at least one of an adapter, a pole, a nut and a housing.

In an embodiment of the invention, the adapter comprises a cap and a base.

In an embodiment of the invention, the base is provided with serrations or teeth, which are formed as counterparts to serrations or teeth located at a top of the pole.

In an embodiment of the invention, the pole is circular in shape at a top of the pole and D-shaped at a bottom of the pole.

In an embodiment of the invention, the housing is provided with a plurality of petals at a top of the housing.

In an embodiment of the invention, an exterior surface of the pole is threaded to match inner facing, threaded surfaces of the plurality of petals.

In an embodiment of the invention, the nut is cylindrical in shape and is disposed around the pole, where the pole passes through a center of the nut.

In an embodiment of the invention, the nut is configured with a threaded inner surface.

In an embodiment of the invention, a top of the housing is provided with threading which is configured as a counterpart to the threaded inner surface.

In an embodiment of the invention, the threaded inner surface is tapered.

In an embodiment of the invention, at least a portion of the housing is D-shaped on the inside to match the D-shape of the pole, such that the pole is slidable within the housing but not rotatable around a longitudinal axis of the pylon assembly.

In an embodiment of the invention, the foot portion includes at least one internal rib.

In an embodiment of the invention, the socket exhibits at least one of a 2 petal, a 3 petal, a 4 petal, a symmetric, an asymmetric and a side branch petal shape.

In an embodiment of the invention, the apparatus further comprises a cover attached to the pylon assembly configured with an anatomically correct shape of a lower leg.

There is further provided in accordance with an aspect of the invention, a method of using a prosthetic leg apparatus with a user's stump, comprising: choosing a socket for use with the user's stump; heating the socket to a temperature sufficiently high to make the socket malleable; forming the socket around the user's stump; cooling the socket to a temperature where the socket is rigid; and, connecting at least a pylon assembly to the socket.

In an embodiment of the invention, the method further comprises connecting a foot portion to the pylon assembly.

In an embodiment of the invention, the method further comprises repeating the heating, forming and cooling to re-configure the socket to the user's stump.

There is further provided in accordance with an aspect of the invention, a protective cover of a pylon assembly with a longitudinal axis, comprising: a front piece anatomically shaped and sized like the shin of a healthy human leg; and, a back piece anatomically shaped and sized like a calk of a healthy human leg.

In an embodiment of the invention, the protective cover further comprises at least one clip provided to the front piece and at least one clip provided to the back piece, which together form a channel in which the pylon assembly is located when the protective cover is removably attached to the pylon assembly.

In an embodiment of the invention, the protective cover further comprises a plurality of interchangeable and different sized and shaped front and back pieces.

There is further provided in accordance with an aspect of the invention, a method of tool-less installation of a prosthetic leg assembly, comprising: placing a nut around a pylon; moving the nut towards external threading of an ankle thread interface or a pylon thread interface; screwing the nut onto and past a first portion of the external threading until internal threading of the nut rests in a gap or the ankle thread interface or the pylon thread interface; applying pressure to petals of the ankle thread interface or the pylon thread interface with an internal tapering of the nut by screwing the nut onto a second portion of external threading of the ankle thread interface or the pylon thread interface.

In an embodiment of the invention, the method further comprises unscrewing the nut from the external threading of the ankle thread interface or the pylon thread interface.

There is further provided in accordance with an aspect of the invention, a socket for use with a user's stump and a prosthetic leg apparatus, comprising: at least one petal, constructed of a thermoplastic material, configured to wrap at least partially around the user's stump, wherein the at least one petal has at least one slot and hole therethrough.

In an embodiment of the invention, the socket further comprises at least one strap inserted through the at least one slot and hole.

In an embodiment of the invention, the socket comprises a plurality of symmetrical petals.

In an embodiment of the invention, the socket comprises a plurality of non-symmetrical petals.

In an embodiment of the invention, the at least one petal has a side branch petal.

In an embodiment of the invention, the at least one of Orthitrans, Delrin, acrylonitrile butadiene styrene, nylon, silicone, polystyrene, and polypropylene.

In an embodiment of the invention, the thermoplastic material activates between 50°-100° C.

There is further provided in accordance with an aspect of the invention, a method of tool-less installation of a prosthetic leg assembly, comprising: fitting a cap to a socket by placing at least one snap fit prong or snap fit post through at least one of a slot and a hole; and, reversibly snapping a base onto the at least one snap fit prong or snap fit post of the cap, trapping the socket therebetween.

In an embodiment of the invention, the method further comprises attaching the base to a pole of a pylon assembly of the prosthetic leg assembly.

In an embodiment of the invention, the attaching is performed using at least one snap fit prong or at least one snap fit post.

In an embodiment of the invention, the method further comprises unsnapping the base using a notch located on at least one of the cap, the base and the pole.

There is further provided in accordance with an aspect of the invention, a foot portion of a prosthetic leg assembly, comprising: a closed-loop main body shaped with a general outline of a human foot; and, an opening provided on the top of the main body configured as a counterpart to a bottom of a housing of a pylon assembly of the prosthetic leg assembly.

In an embodiment of the invention, the foot portion further comprises at least one internal rib extending from a top of the main body to a bottom of the main body.

In an embodiment of the invention, the foot portion further comprises at least one circumferential rib extending around an inner surface of the main body.

In an embodiment of the invention, the foot portion is configured with a plurality of different sizes.

In an embodiment of the invention, the foot portion is configured with a plurality of different levels of traction on the exterior of a bottom of the main body.

In an embodiment of the invention, the foot portion is configured with a plurality of different levels of resiliency.

In an embodiment of the invention, a bottom of the main body is convex.

In an embodiment of the invention, a heel of the main body is rounded.

In an embodiment of the invention, a heel of the main body is positioned a longitudinal distance away from the opening.

In an embodiment of the invention, a front of the main body is curved.

In an embodiment of the invention, the foot portion is provided with a tool-less interface between the opening and the housing.

There is further provided in accordance with an aspect of the invention, a prosthetic leg assembly system, comprising: a plurality of foot portions, wherein each foot portion of the plurality is configured differently and is interchangeable with any one of the plurality of foot portions.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWING(S)

Some embodiments are herein described, by way of example only, with reference to the accompanying drawings. With specific reference now to the drawings in detail, it is stressed that the particulars shown are by way of example, are not necessarily to scale, and are for purposes of illustrative discussion of embodiments of the invention. In this regard, the description taken with the drawings makes apparent to those skilled in the art how embodiments of the invention may be practiced.

In the drawings:

FIG. 1 is a perspective view of a prosthetic leg apparatus, in accordance with some embodiments of the invention;

FIG. 2 is a perspective view of a prosthetic leg apparatus without a socket, in accordance with some embodiments of the invention;

FIG. 3 is a partially exploded view of a prosthetic leg apparatus without a socket, in accordance with some embodiments of the invention;

FIGS. 4A-4B are top and perspective views, respectively, of a socket, in accordance with some embodiments of the invention;

FIGS. 5A-5B are top and perspective views, respectively, of a socket, in accordance with some embodiments of the invention;

FIGS. 6A-6B are top and perspective views, respectively, of a socket, in accordance with some embodiments of the invention;

FIGS. 7A-7E are top, front, side, and perspective views, respectively, of a socket, in accordance with some embodiments of the invention;

FIGS. 8A-8B are top and perspective views, respectively, of a socket, in accordance with some embodiments of the invention;

FIGS. 9A-9B are top and perspective views, respectively, of a socket, in accordance with some embodiments of the invention;

FIGS. 10A-10B are top and perspective views, respectively, of a socket, in accordance with some embodiments of the invention;

FIGS. 11A-11B are top and perspective views, respectively, of a socket, in accordance with some embodiments of the invention;

FIGS. 12A-12B are top and perspective views, respectively, of a socket, in accordance with some embodiments of the invention;

FIGS. 13A-13B are top and perspective views, respectively, of a socket, in accordance with some embodiments of the invention;

FIGS. 14A-14B are top and perspective views, respectively, of a socket, in accordance with some embodiments of the invention;

FIG. 15 is a perspective view of a formed socket with straps, in accordance with some embodiments of the invention;

FIG. 16 is a perspective view of a formed socket with straps, in accordance with some embodiments of the invention;

FIGS. 17A-17B are top and bottom, respectively, perspective views of a serrated disc of an adapter, in accordance with some embodiments of the invention;

FIGS. 18A-18C are views of a pole, in accordance with some embodiments of the invention;

FIGS. 19A-19B are a top view and a cross-sectional view of the bottom of the pole of FIGS. 18A-18C, in accordance with some embodiments of the invention;

FIGS. 20A-20B are perspective and cross-sectional views, respectively, of a housing;

FIGS. 21A-21B are perspective and cross-sectional views, respectively, of a nut, in accordance with some embodiments of the invention;

FIGS. 22A-22B are perspective and side views, respectively, of a foot portion, in accordance with some embodiments of the invention;

FIG. 23 is a perspective view of a socket mounted on an adapter, in accordance with some embodiments of the invention;

FIG. 24 is a partially exploded view showing a bottom of a housing interfacing with a foot portion, in accordance with some embodiments of the invention;

FIG. 25 is an illustration of a user shaping a socket, in accordance with some embodiments of the invention;

FIG. 26 is a flowchart of a method of using a prosthetic leg apparatus, in accordance with some embodiments of the invention;

FIGS. 27A-27C are a side, cross-sectional and bottom views, respectively, of the pole with an adapter attached thereto, in accordance with some embodiments of the invention;

FIG. 28 is an illustration of a user wearing a prosthetic leg apparatus, in accordance with some embodiments of the invention;

FIG. 29 is a perspective view of an ankle thread interface, in accordance with some embodiments of the invention;

FIG. 30 is a perspective view of a pylon thread interface, in accordance with some embodiments of the invention;

FIGS. 31A-31B are a perspective view and a cross-sectional view, respectively, of a nut, in accordance with some embodiments of the invention;

FIGS. 32A-32D are progressive cross-sectional views of a tool-less installation method, in accordance with some embodiments of the invention;

FIGS. 33A-33E are a front, side, back, perspective and top views, respectively, of a cover, in accordance with some embodiments of the invention;

FIGS. 34A-34E show the cover attached to the prosthetic leg apparatus, where the views correspond to those of FIGS. 33A-33E, in accordance with some embodiments of the invention;

FIGS. 35A-35B are side and cross-sectional views, respectively, of a prosthetic leg apparatus, in accordance with some embodiments of the invention; and,

FIGS. 36A-36B are front and cross-sectional views, respectively, of a prosthetic leg apparatus, in accordance with some embodiments of the invention.

### DESCRIPTION OF SPECIFIC EMBODIMENTS OF THE INVENTION

The present invention, in some embodiments thereof, relates to the health care field and, more particularly, but not exclusively, to prosthetic limbs.

Before explaining at least one embodiment of the invention in detail, it is to be understood that the invention is not necessarily limited in its application to the details of construction and the arrangement of the components and/or methods set forth in the following description and/or illustrated in the drawings. The invention is capable of other embodiments or of being practiced or carried out in various ways.

According to some aspects of the invention, prosthetic leg apparatuses described herein are optionally configured to be highly adjustable (e.g. by the user). For example, the apparatuses described herein are configured/designed to be customized by the user and/or self-adjusted in a non-laboratory or non-specialized setting, using reusably thermosetting materials and/or tool-less construction and/or adjustable size/configuration. This is in contrast to how prosthetics are customized for a user today, which is to say, in a long and drawn-out (often taking days to weeks), expensive, precision fitting process typically performed in a laboratory with high cost, specialized technicians and high precision machinery.

According to some aspects of the invention, prosthetic leg apparatuses described herein are optionally configured to be inexpensive relative to conventional prosthetic apparatuses. For example, being constructed of relatively inexpensive materials and/or construction/customization processes, including obviating the need for expensive equipment for fitting the prosthetic to the user.

According to some aspects of the invention, the prosthetic leg apparatuses described herein are used by children, particularly those in third-world and/or impoverished living situations, who are subjected to sub-standard health care and/or armed-conflict related injuries, such as those sustained from land mines. Various embodiments of the prosthetic leg apparatuses described herein are configured to be used for a long duration and adjusted/adapted by/for these children relatively easily and/or in a non-laboratory setting over time as they grow. In some aspects, overall cost savings are realized simply by configuring the prosthetic leg apparatuses to “grow” along with the child user, that is, the child can use the same or mostly the same apparatus throughout different stages of life, as the child grows up.

According to some aspects of the invention, prosthetic leg apparatuses are provided which are modular, comprising multiple components such as a socket, straps, a pylon assembly and/or a foot, each of which is interchangeable with other versions of the same component in the overall prosthetic leg apparatus.

According to some aspects of the invention, embodiments of a pylon assembly of the prosthetic leg apparatus are provided with a pylon which nests within a housing wherein the pylon is reversibly securable within the housing at different adjustable heights. Optionally, the pylon assembly utilizes a screwing nut for reversibly securing the pylon into the housing.

According to some aspects of the invention, embodiments of a foot portion of the prosthetic leg apparatus are configured for durability, to be light weight, and responsive to the various gait phases of walking movement (forward and backward). For example, constructing the foot portion from plastic materials contributes to the light weight characteristic. Certain plastic materials which exhibit elastic properties and/or are not rigid are utilized for reducing the impact of movement on the user and/or to provide energy storage and/or return. In some embodiments, a plurality of foot portions are offered which are modular and/or interchangeable, for example being configured in different sizes and/or shapes and/or for different purposes (e.g. walking, running, sports, varying traction, varying resiliency).

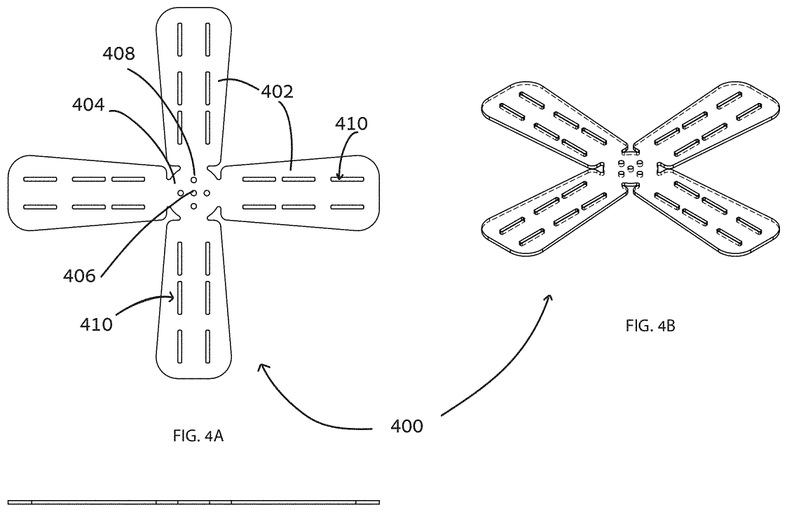

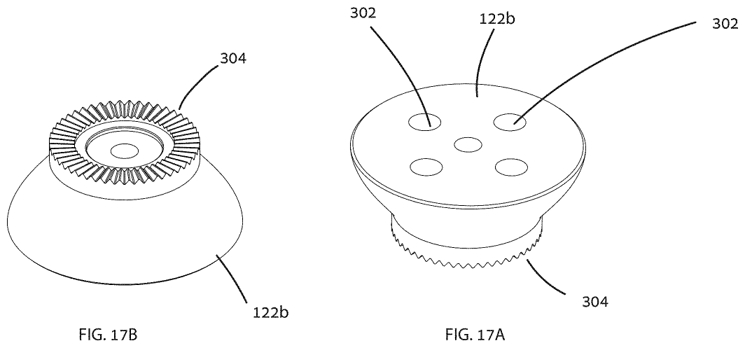

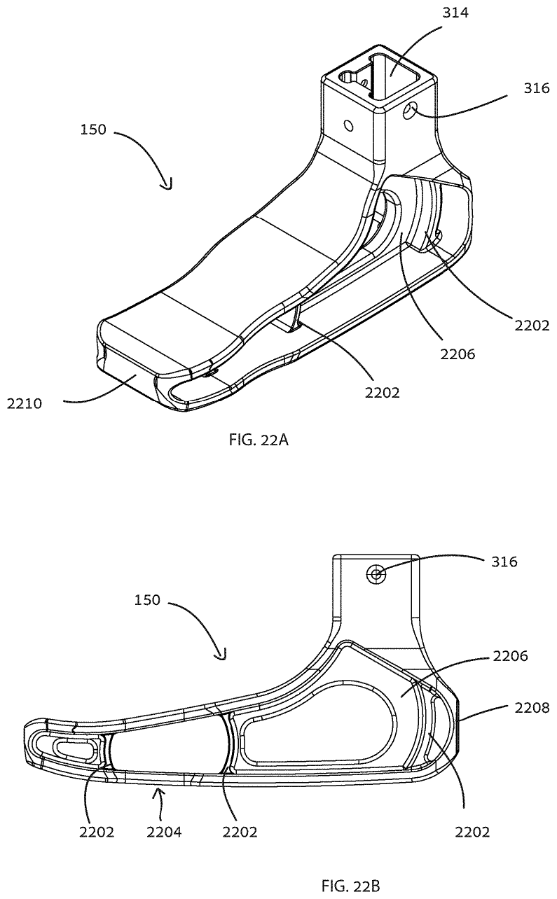

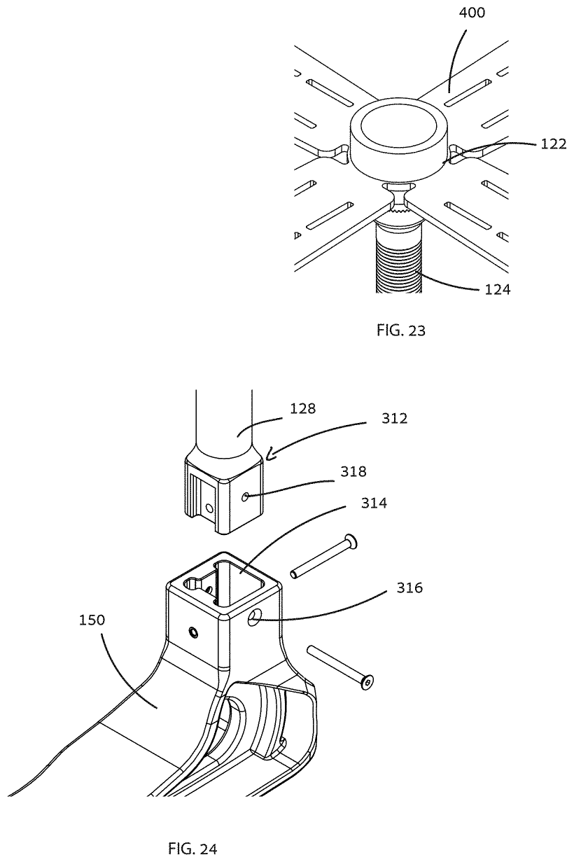

Generally, different embodiments of a prosthetic leg apparatus are described wherein the apparatuses, in some embodiments, comprise at least a socket, a pylon assembly and a foot portion. Socket embodiments, and appurtenant strap embodiments, are described in particular with respect to FIGS. 4A-16, 23 and 25. Pylon assembly embodiments, and components of the pylon assembly, are described in particular with respect to FIGS. 17A-21B, 27A-27C, 29, 30, 31A-31B, 32A-32D, 33A-33E, and 34A-34E. Foot portion embodiments are described in particular with respect to FIGS. 22A-22B and 24.

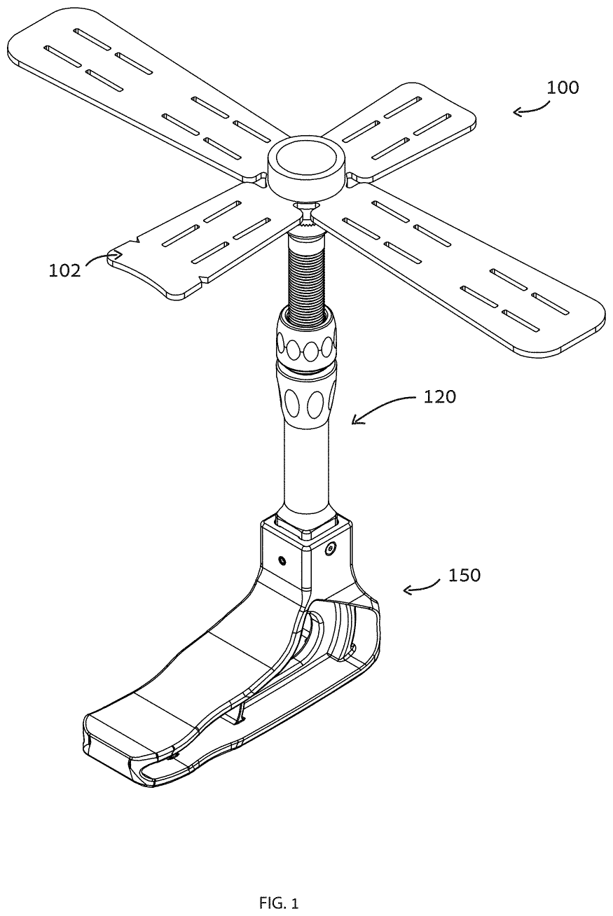

Referring now to the drawings, FIG. 1 is a perspective view of prosthetic leg apparatus 100, according to an aspect. The prosthetic leg apparatus 100 comprises a socket 102 (straps not shown), a pylon assembly 120, and a foot portion 150.

In some embodiments, the socket 102 is used to connect the user's stump to the prosthetic leg apparatus 100, wherein the user inserts a remaining portion of the user's leg into the socket and where the socket is attached, optionally removably, to the pylon assembly 120. In some embodiments, the socket 102 is constructed of a pre-shaped layout made of material that may be formed to the shape of the user's stump. For example, the socket 102 is built of at least one of a thermoplastic material (e.g. various OrfitransTM materials manufactured by Orfit Industries, Delrin®, ABS) and/or at least one special fabric/material (e.g. nylon, polypropylene, silicone, polystyrene). Other materials which could be used include Celcon, Ramtal, Duracon, Kepital and Hostaform. Optionally, reinforcement is provided by fiberglass or the like. Thermoplastic materials are plastic materials that change their elastic, adhesive, plasticity and flexibility characteristics when exposed to high temperature and can be shaped at this phase to a dedicated structure. In some embodiments, the socket 102 is offered in a spectrum, large or small, of sizes to be selected for use depending on the needs of the individual user. In some embodiments, the socket 102 is anywhere from 1 mm-5 mm in thickness. In some embodiments, the activation temperature ranges between 50°-100° C. In some embodiments, the heated thermoplastic is contactable by hand within a minute. It should be understood that these numbers are given by way of example only and that depending on the material used, the intended purpose and/or the user these characteristics could vary.

In practice, the user would heat the socket 102, for example by submerging it in hot or boiling water, to a temperature where the socket becomes temporarily malleable. The user then holds the malleable socket to the user's stump to form it around the stump, thereby customizing the socket to the user's own anatomy. The malleability of the socket diminishes once the socket begins to cool down and the newly formed socket remains in the shape as customized by the user. The socket can be repetitively customized, for example as the user grows and/or for comfort, through repeated cycles of heating, forming and cooling.

By using a customizable design, it is conceived that the socket 102 will fit any user in a more usable fashion and for over a longer period of time (since it can be repetitively customized as the user's needs change) than conventional prosthetic attachment components, since the socket 102 can be self-fitted/customized, repeatedly, without the need of special tools, laboratory setting and/or expensive machinery. Additional advantages include the ability to customize without the need of any special tools or additional materials and the ability of the user being able to perform the customization solo (e.g. without help from any additional person or a medical professional).

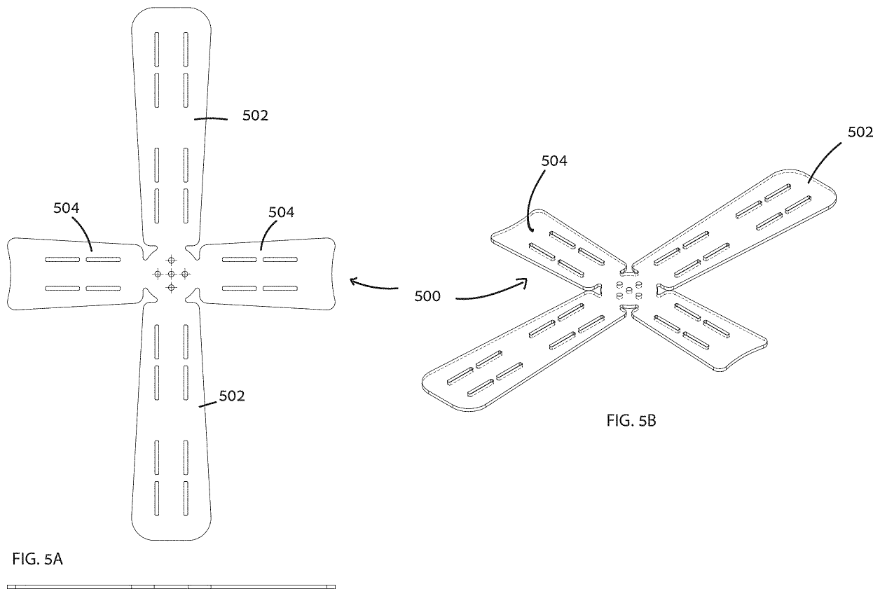

It should be understood that while the socket 102 shown in FIG. 1 corresponds to the socket 500 shown in FIGS. 5A-5B, any socket could be used, particularly the other socket embodiments described herein. In some embodiments, the socket 102 is used in conjunction with at least one strap which passes through the socket 102 and which assists with securing the socket 102 to the user. Additional details of the socket 102, generally and particularly, are described below. It should also be understood that while different parts of the apparatus 100 are described separately herein, some or all of the parts could be a unified part, for example the foot and pylon could be a single part, or the socket and pylon could be a single part.

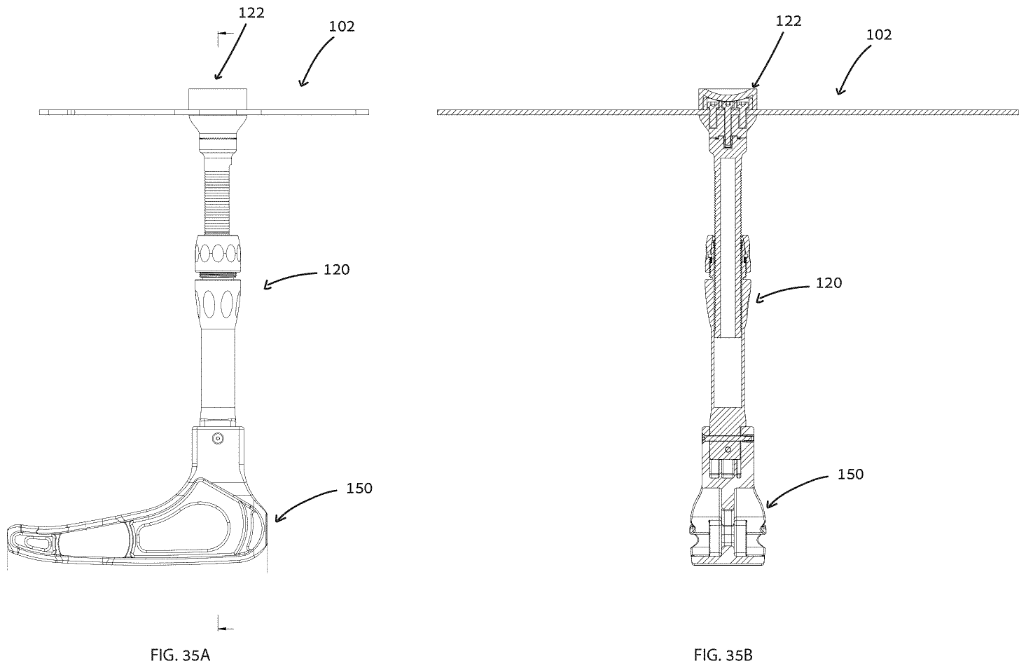

FIGS. 35A-35B are side and cross-sectional views, respectively, of the prosthetic leg apparatus 100. The prosthetic leg apparatus 100 is shown in FIG. 35A from the side, including the socket 102 (straps not shown), the adapter 122, the pylon assembly 120, and the foot portion 150. FIG. 35B is a cross-section indicated by E-E in FIG. 35A of prosthetic leg apparatus 100.

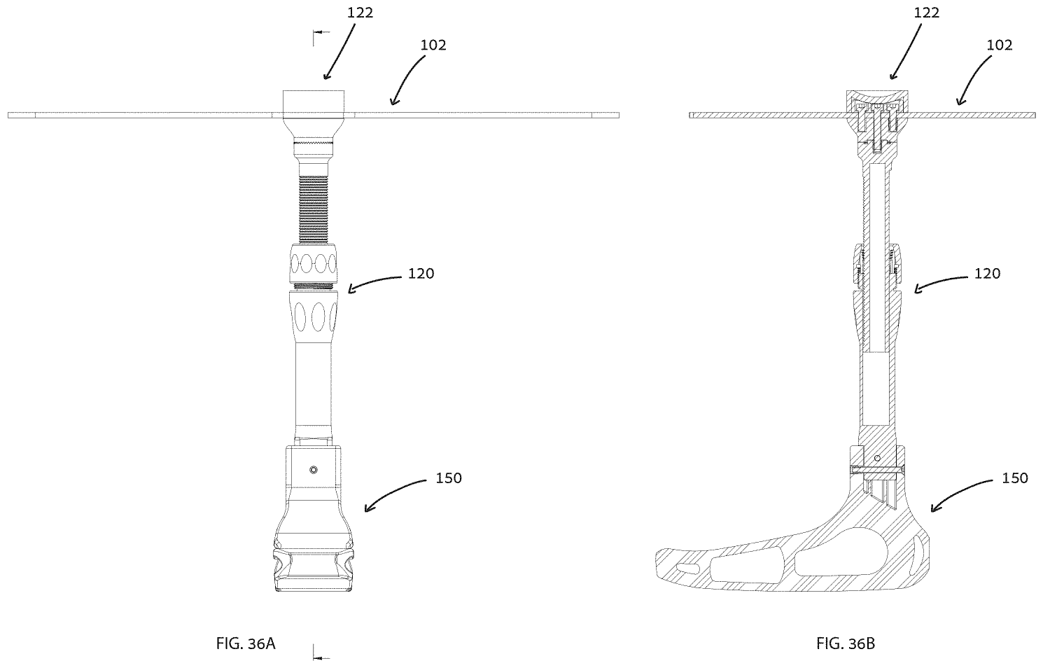

FIGS. 36A-36B are front and cross-sectional views, respectively, of the prosthetic leg apparatus 100. The prosthetic leg apparatus 100 is shown in FIG. 36A from the front, including the socket 102 (straps not shown), the adapter 122, the pylon assembly 120, and the foot portion 150. FIG. 36B is a cross-section indicated by D-D in FIG. 36A of prosthetic leg apparatus 100.

FIG. 2 is a perspective view of prosthetic leg apparatus 100 without the socket 102, for enhanced clarity. More clearly shown are the pylon assembly 120, which is a collection of components, and the foot portion 150.

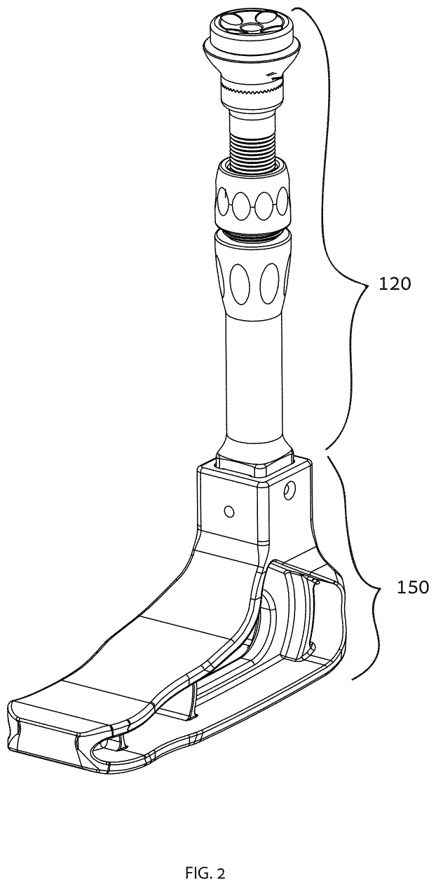

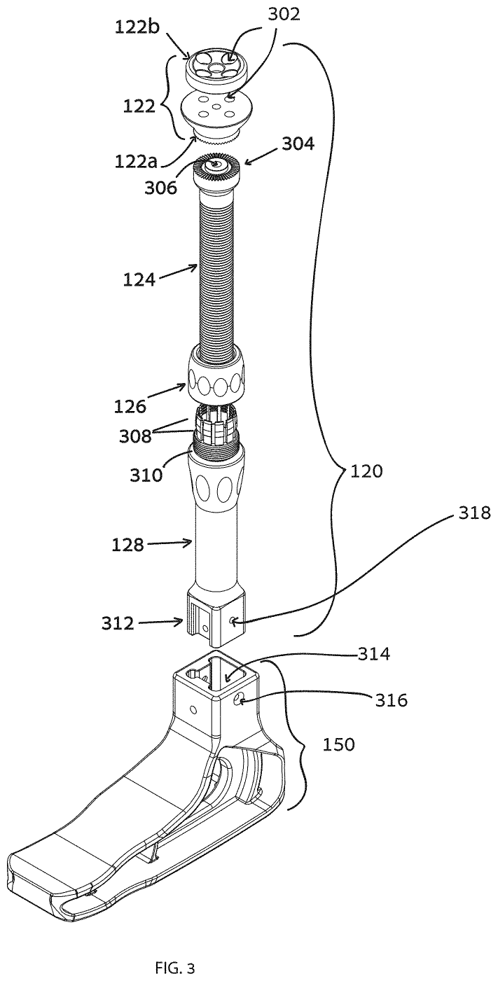

FIG. 3 is a partially exploded view of prosthetic leg apparatus 100 without the socket 102, in an embodiment. In some embodiments, the pylon assembly 120 is structurally equivalent to the shin of a healthy leg and is designed to support the weight of the user during gait. In some embodiments, the pylon assembly 120 comprises, but is not necessarily limited to or is required to have, an adapter 122, a pole 124, a nut 126 and a housing 128.

It is conceived that the pylon assembly 120 is adjustable and/or assemble-able without the need of special tools. In an embodiment, the pole 124 and the housing 128 together function as a telescoping structure that can be lengthened or shortened in their longitudinal axis to modify or set a desired length of the prosthetic leg apparatus 100, for example to make the prosthetic leg apparatus 100 longer as a child user grows taller. In some embodiments, the pylon assembly is adjustable in height (longitudinal axis) from 225 mm-400 mm, as an example.

In some embodiments, the adapter 122 comprises a cap 122a and a base 122b. Base 122b is shown in more detail with respect to FIGS. 17A-17B. The cap 122a and the base 122b are configured with at least one hole 130 threaded and/or configured for receipt of a screw therein/therethrough. Cap 122a and base 122b are shown in FIG. 3 with 5 such holes 302 each, which correspond to each other from the cap 122a to the base 122b.In an embodiment, a bottom of the base 122b (the side of the base 122b facing the pole 124) is provided with serrations or teeth which are formed as counterparts to serrations or teeth 304 located at a top of the pole 124. These counterpart serrations/teeth provide the ability to fine tune direction of the socket/stump with the direction of foot, by turning the pole 124 relative to the base 122b where the serrations/teeth hold the desired orientation in place.

In some embodiments, the connection between the cap 122a and the base 122b is configured to be tool-less for attachment and detachment. For example, the cap 122a could be provided with at least one snap fit prong, which fit into counterpart holes and/or slots in the base 122b, trapping/bracketing the socket 102 therebetween. In some embodiments, the base 122b is configured to connect to the pole 124 in a tool-less fashion, for example being provided with at least one snap fit fin and/or post and/or prong designed as a counterpart to a female portion located at the top of the pole 124. Optionally, male and female parts are reversed and/or alternate. Optionally, a notch is provided to at least one of the cap 122a, the base 122b, and the pole 124 to facilitate detaching the snap fit connection.

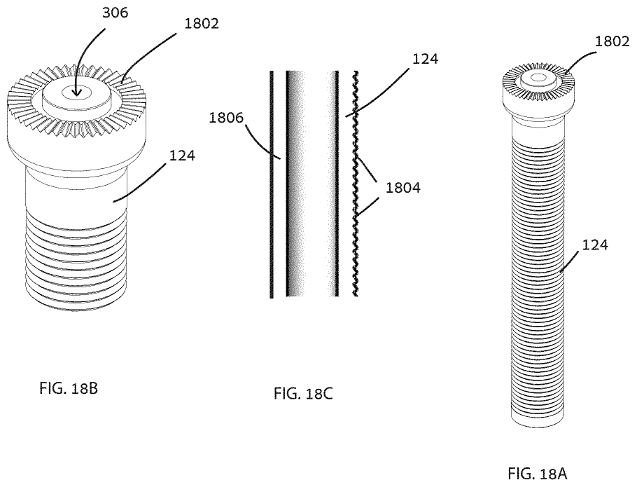

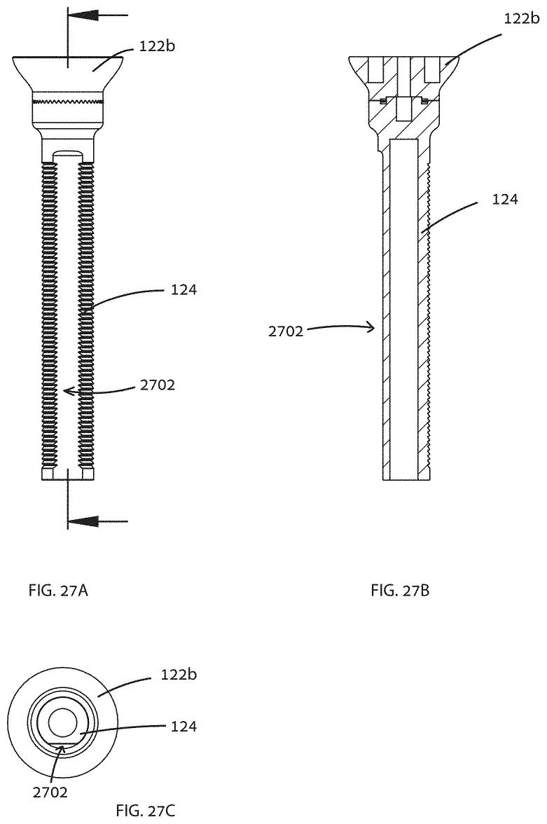

In some embodiments of the invention, the top of the pole 124 is provided with a hole 306 configured for receipt of a screw therein, for example the same screw which passes through the center of the cap 122a and the base 122b (in embodiments which screw together instead of or additionally to snap fit). The top of the pole 124 is also formed as a circular shape to match a circular shape of the base 122b, in an embodiment. In some embodiments, although the top of the pole 124 is circular in shape, the remainder of the pole 124 is configured with a D-shaped cross-section, such as shown in FIGS. 27B and 27C, for example to prevent the pole 124 from rotating (because it is round) within the housing 128 when the pole 124 is inserted into the housing 128. In some embodiments of the invention, the exterior surface of the pole 124 is threaded to match inner facing threaded surfaces of a plurality of petals 308 of the housing 128. In some embodiments, the flat surface 2702 of the D-shape is not threaded, just the curved surface. An embodiment of the pole 124 is shown in more detail in FIGS. 18A-18C.

FIGS. 27A-27C are a side, cross-sectional and bottom views, respectively, of the pole 124 with an adapter 122 attached thereto. FIG. 27A shows the flat surface 2702 of the D-shaped pole 124. FIG. 27B is a cross-section along line A-A of FIG. 27A showing the flat surface 2702 from the side. FIG. 27C is a bottom view of the pole 124 which shows the flat surface 2702 defining a D-like shape of the pole 124 (although it is a backwards “D” as oriented in this particular view).

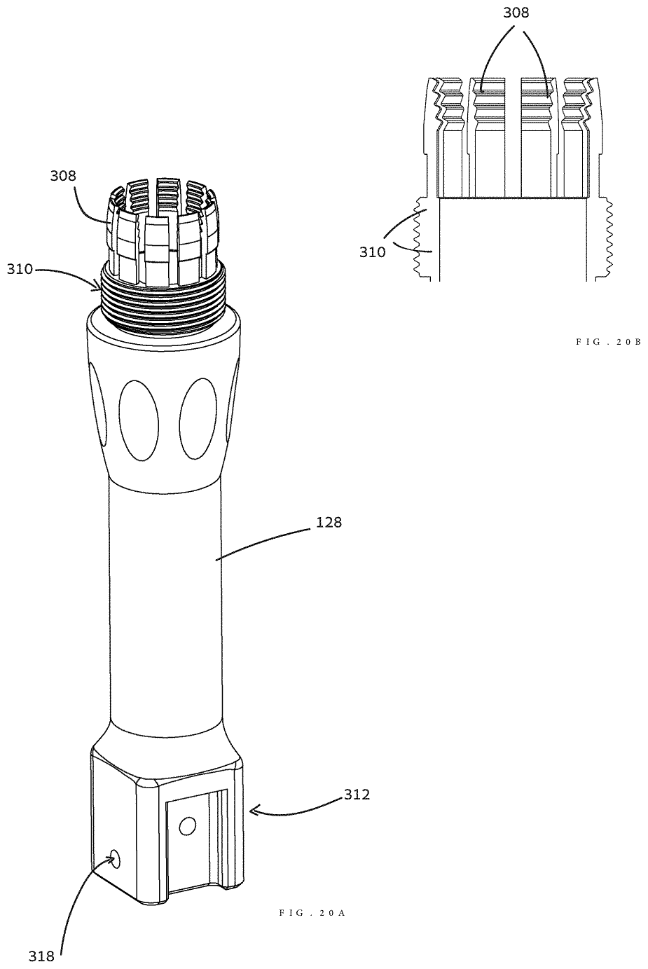

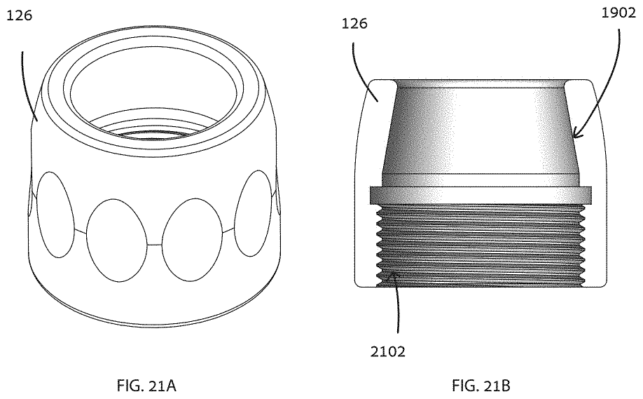

In some embodiments of the invention, the nut 126 is cylindrical in shape and is disposed around the pole 124, with the pole 124 passing through the center of the nut 126. The nut 126 is configured with a threaded inner surface (shown in more detail in FIG. 21B) which is designed as a counterpart to threading 310 located on the outside of the top of the housing 128. In practice, the nut 126 is screwed down onto the housing 128 to connect the pole 124 to the housing 128. In some embodiments, the nut 126 is tapered on the inside to apply pressure to the petals 308 when the nut 126 is screwed onto the housing 128. This tapering is shown in more detail in FIG. 21B.

The housing 128 structurally connects the pole 124 to the foot portion 150. As described above, the housing 128 includes a plurality of petals 308, which on an inner surface are threaded to act as a counterpart to threading on the exterior surface of the pole 124. In an embodiment, the housing 128 is hollow on the inside and is D-shaped to match the D shape of the pole 124, such that the pole 124 is slidable within the D-shaped hollow of the housing 128. A bottom 312 of the housing 128 is shaped to interface with the foot portion 150, in some embodiments. Optionally, the bottom 312 is given a characteristic shape which matches with a counterpart characteristic opening 314 in the foot portion 150 which defines the orientation of the housing 128 when the housing 128 is inserted into the foot portion 150. In some embodiments, at least one screw hole 316 is defined in the foot portion 150 with a corresponding hole 318 in the bottom 312 of the housing such that a screw (not pictured) can be used to reversibly attach the housing 128 to the foot portion 150.

As described elsewhere herein, the pole 124 and the housing 128 act in concert as a telescoping unit to lengthen or shorten the prosthetic leg apparatus 100. In an embodiment, the total length is determined according to the needs and/or comfort of the user. For example, as a child user grows, the prosthetic leg apparatus 100 is lengthened to accommodate for and balance the increased length of the user's healthy leg. The D-shape of the pole 124 and the corresponding D-shape of the hollow inside the housing 128 prevent rotation of the pole 124 when the pole 124 is at least partially inserted into the housing 128. Adjusting the length is performed by a ratchet action mechanism, using partial engagement/interaction between the threaded petals 308 of the housing 128 and the threaded exterior surface of the pole 124. In an embodiment, reversible locking of the pole 124 with respect to the housing 128 is performed by screwing the nut 126 onto the threading 310 of the housing 128. As the nut 126 is screwed tighter, the petals 308 of the housing 128 flex towards the pole 124 and the threading on the inside surface of the petals 308 interlocks with the threading of the exterior surface of the pole 124, thereby preventing further up and down (i.e. lengthening and shortening) movement of the pole 124 with respect to the housing 128.

In some embodiments, some form of aesthetic and/or protective cover is added to the pylon assembly 120 to provide a proper anatomic shape to the prosthetic leg apparatus 100. FIGS. 33A-33E are a front, side, back, perspective and top views, respectively, of a cover 3300, in accordance with an embodiment of the invention. FIG. 33A shows the cover 3300 from the front, prominently displaying a front piece 3302 which would be loosely analogous to the shin of a healthy individual. It should be understood that the cover 3300 could be provided in a plurality of interchangeable components exhibiting different sizes and/or different shapes. It is conceived that a user of the cover 3300 could, over time, use larger and larger cover sizes and/or shapes as the user grows and/or could desire differently configured cover shapes and/or sizes depending on different activities being performed.

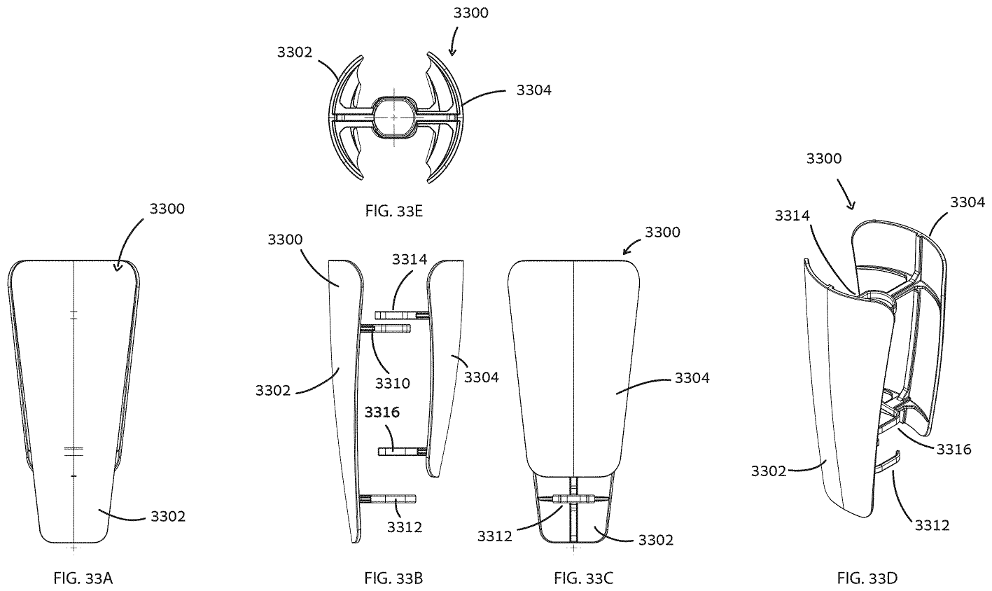

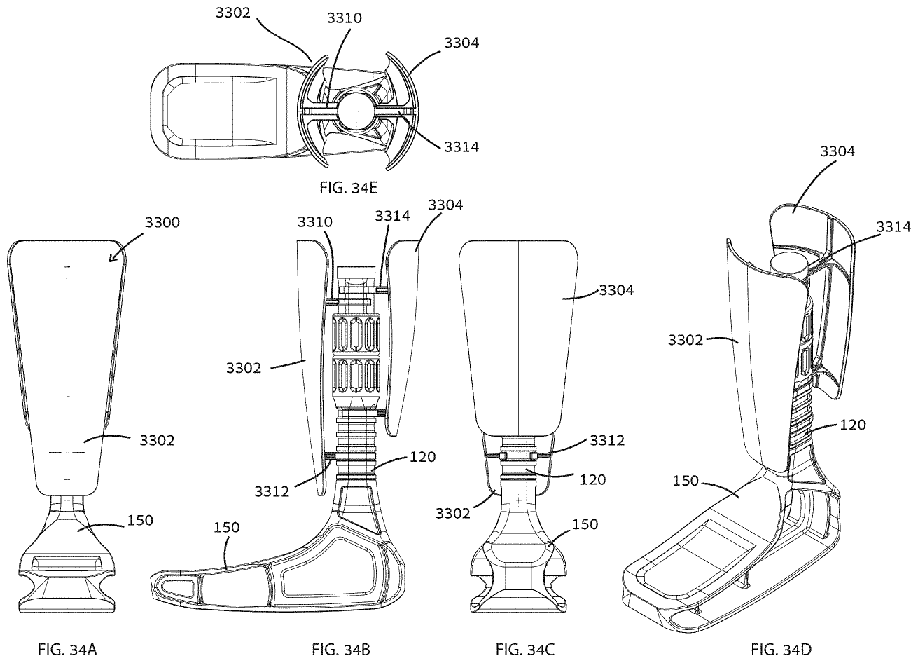

FIG. 33B is a side view of a cover 3300 including a back piece 3304, loosely analogous to a healthy individual's calf muscles, and also showing a plurality of front clips 3310, 3312 (corresponding to upper and lower portions of the front piece) and back clips 3314, 3316 (corresponding to upper and lower portions of the back piece). In an embodiment, the clips 3310, 3312, 3314, 3316 are configured to snap onto the prosthetic leg apparatus 100, for example at various locations along the longitudinal axis of pylon assembly 120 (such as shown in FIG. 34B, inter alia).

FIG. 33C is a back view of the cover 3300, prominently displaying the back piece 3304 and also partly showing an inside portion of the front piece 3302 (where the lower, front clip 3312 is seen).

FIG. 33D is a perspective view, from the front, of the cover 3300. This view of the cover 3300 shows the upper and lower back clips 3314, 3316 connected to the back piece, for example by being molded in unitary construction with the back piece 3304. It should be understood that the clips 3310, 3312, 3314, 3316 could be attached to the front and back pieces in virtually any manner.

FIG. 33E shows the cover 3300 from the top, and in particular, illustrates how the clips 3310, 3312, 3314, 3316 collectively form channel in which the pylon assembly 120 is located when the cover 3300 is used with the prosthetic leg apparatus 100. FIGS. 34A-34E show the cover 3300 attached to the prosthetic leg apparatus 100, where the views correspond to those of FIGS. 33A-33E.

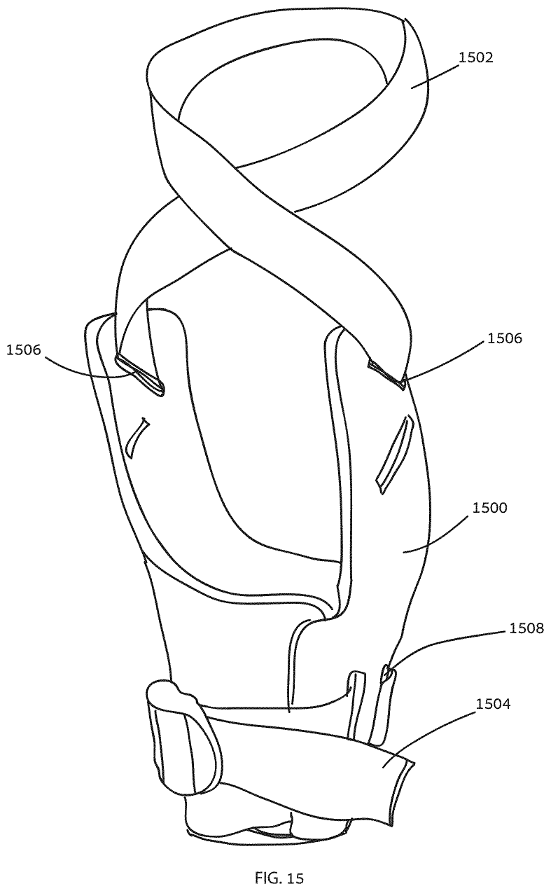

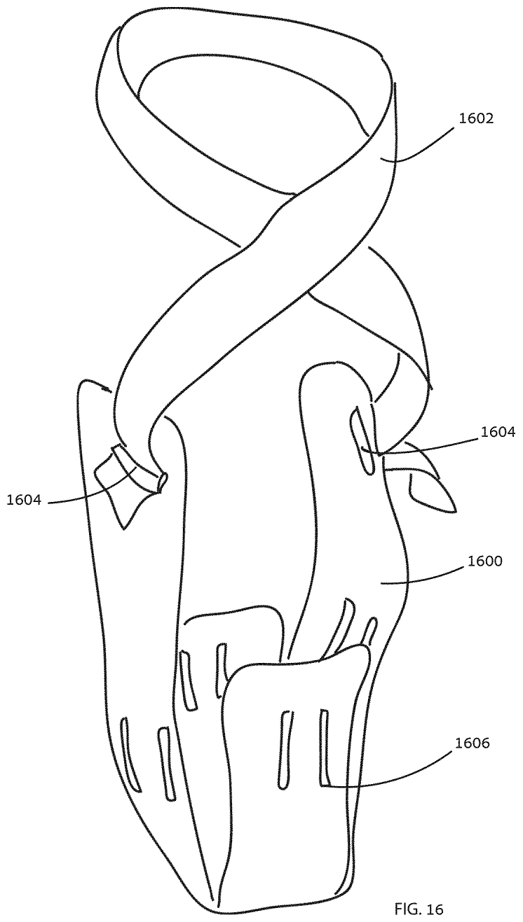

FIGS. 4A-4B are top and perspective views, respectively, of a socket 400. As described elsewhere herein, sockets used with prosthetic leg apparatus 100 are designed to be self-adjusted/fitted by the user, optionally at home, for example as shown in FIG. 25 where a user is folding a socket into a configuration to be used as a part of the prosthetic leg apparatus 100. FIG. 28 shows a user wearing a prosthetic leg apparatus 100, with a socket 2800 already fitted to the user's stump and straps 2802 (straps described in more detail, below). FIG. 4A shows socket 400 with a configuration including 4 equally sized petals 402 extending from a center 404 which connects the petals 402. The center 404 is provided with at least one hole 406 for attaching the socket 400 to the top of the pole 124, optionally as described elsewhere herein by tightening a screw through the hole 406 in the socket 400 through the adapter 122 to the hole 306 in the pole 124. In some embodiments, at least one additional hole 408 is provided to further secure the socket 400 to the adapter 122, where the adapter 122 is provided with corresponding holes for this purpose. At least one cut 410 is provided to the socket 400 to provide ventilation and/or a location for threading a strap therethrough, such as shown in FIGS. 15 and 16.

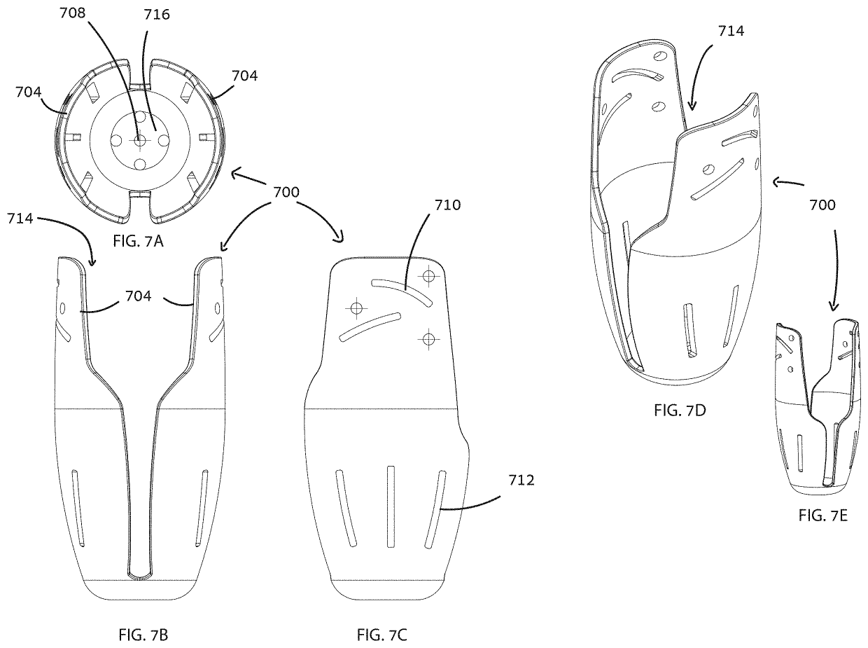

The socket 400 could be offered in a flattened configuration such as shown in FIGS. 4A-4B, or pre-formed to a generally useful shape, such as shown with socket 600 in FIGS. 7A-7E. In some embodiments, flattened and/or pre-formed sockets are adapted by the user to the user's individual anatomy as needed. In some embodiments, at least a part of the socket 400 is provided with an adhesive and/or tacky substance on a surface facing the user to better and temporarily fixate the socket 400 to the user's stump. In some embodiments, at least a part of the socket 400 facing the user is provided with a cushioning substance, like padding, rubber or silicone, to provide additional comfort to the user wearing the prosthetic leg apparatus 100. The socket 400 could be offered including at least one strap, optionally including at least one strap already threaded through at least one cut 410. In some aspects, the thermoplastic material is provided with holes for aeration and/or with a sticky or tacky characteristic that helps during the forming process (for example, after being warmed).

In an embodiment, socket 400 is positioned in the middle of the two components of the adapter 122, the cap 122a and the base 122b, wherein a screw which is used to attached the socket 400 to the pole 124 passes first through the cap 122a, then the socket 400, then the base 122b, then into the top of the pole 124. Optionally, a cushioning cup is placed over the cap 122a to make the interface between the user's stump and the apparatus 100 more ergonomic.

It should be understood that features and/or options described with respect to any of the sockets herein could be applied to any of other socket, including other sockets described herein, unless otherwise noted.

FIGS. 5A-5B are top and perspective views, respectively, of a socket 500. It can be seen that socket 500 is shaped slightly differently than socket 400, including two elongated petals 502 and two shortened petals 504 with slightly different convexly curved ends. It should be understood that with this alternative configuration, and other different configurations described herein, that when the socket is formed to the user, the formed shape will be different depending on the socket configuration. It is conceived that since different users will have different individual needs, a plurality of socket configurations may be offered to best meet a particular user's needs. For example, socket 500 is configured to allow more freedom of movement of the knee (because of the shorter petals 504) relative to other shapes, like socket 400.

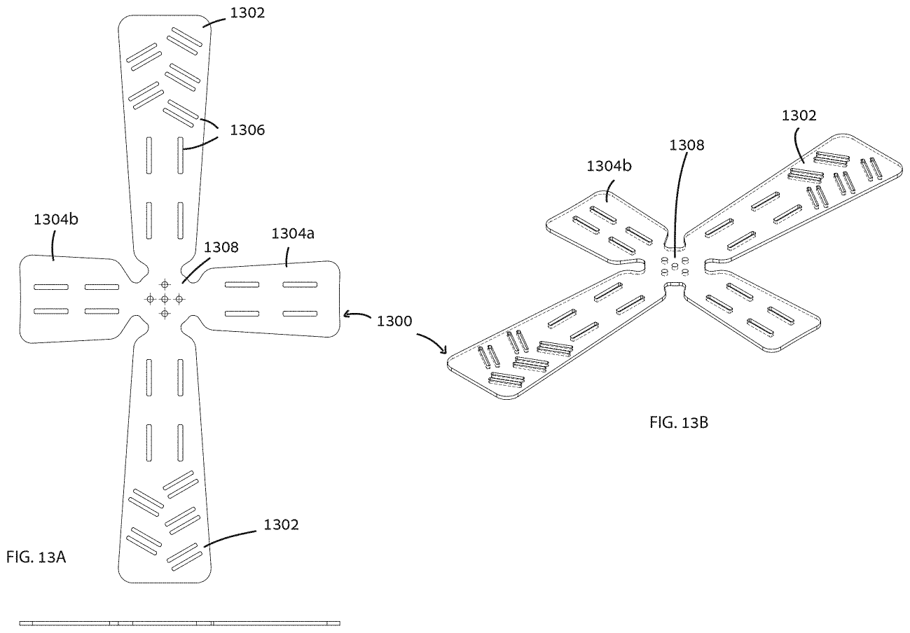

FIGS. 13A-13B are top and perspective views, respectively, of a socket 1300. In an embodiment, socket 1300 is provided with two “long” petals 1302 and two “short” petals 1304a, 1304b, wherein “long” means longer relative to the other two “short” petals 1304a, 1304b. Short petals 1304a, 1304b are intended to be located on the front (petal 1304a) and rear (petal 1304b) of the user's stump when the prosthetic leg apparatus 100 is put on by the user. It is believed that the shorter petals 1304a, 1304b will allow for easier movement of the user's leg than if they were the same length as petals 1302. In some embodiments, the front petal 1304a is intended to be located under the patella and retained to the stump using straps, such as shown and described with respect to other sockets elsewhere herein. In some embodiments, the rear, short petal 1304b is shorter than the front, short petal 1304a to facilitate knee-bending movements.

As with other socket embodiments described herein, at least one cut 1306 is made in the socket 1300 to provide slots for strap insertion and/or ventilation. For example, the socket 1300 can be worn by the user such that at least one cut is disposed below the knee and at least one cut is disposed above the knee, and wherein at least one strap through these cuts provide a slightly different functionality or usefulness (e.g. straps under the knee facilitate knee movement, whereas straps above the knee facilitate knee bending, although these are not mutually exclusive).

In some embodiments, the arrangement of the petals 1302, 1304 is designed to optimize and/or maximize surface area contact of the socket 1300 with the stump, while minimizing overlap of any of the petals 1302, 1304. In some embodiments, at least some overlap of the petals 1302, 1304 is intended and/or acceptable, particularly in embodiments where at least a portion of the petals 1302, 1304 are configured to reversibly adhere to each other to increase socket 1300 strength. In some embodiments, the petals 1302, 1304 are generally configured to taper towards the center 1308 of the socket 1300, such that the upper end of the socket 1300 when formed is wider than the bottom (near the center 1308).

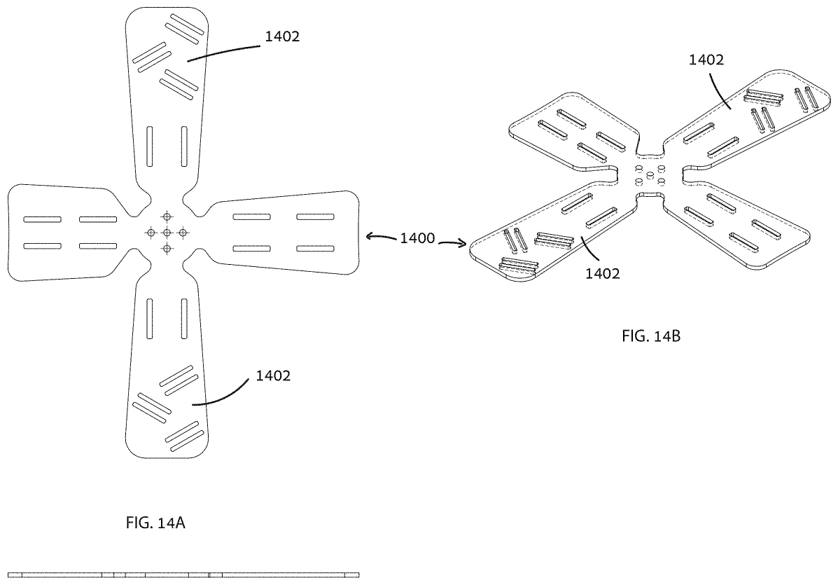

Socket 1400 shown in FIGS. 14A-14B is generally similar to socket 1300, with the “long” petals 1402 being shorter than the long petals 1302 of FIGS. 13A-13B. In an aspect, the varied length of petal could be chosen based on the user stump size.

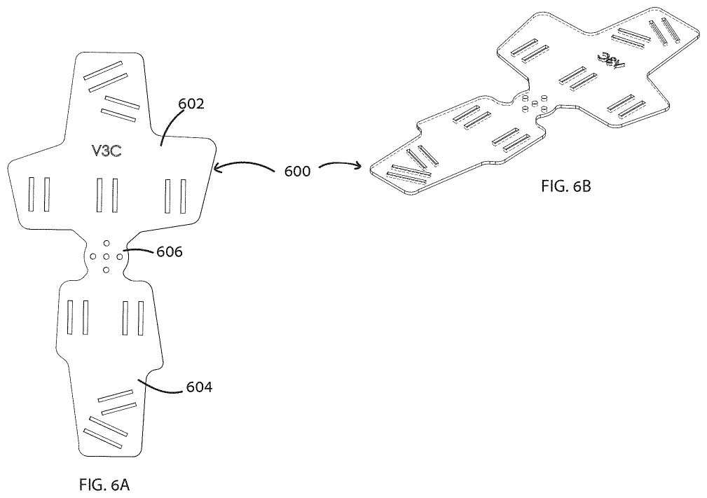

FIGS. 6A-6B are top and perspective views, respectively, of a socket 600. Socket 600 is distinguished from sockets 400, 500 in that there are only two petals, 602, 604 and they are asymmetrical with respect to each other. A center 606 of socket 600 connects first petal 602 to second petal 604. In an aspect, it is believed that the asymmetrical shape of socket 600 will be easier to handle with two hands by a user, in comparison to the shapes of sockets 400, 500, while also providing free movement to the knee joint (when the socket 600 is oriented correctly).

FIGS. 7A-7E are top, front, side, and perspective views, respectively, of socket 700 formed by the heating, forming and cooling cycle described elsewhere herein. In an embodiment, socket 700 is provided with two petals 704 which are formed with a space 714 therebetween for receipt of the user's stump therein. As described elsewhere herein, space 714 is optionally formed by the user heating the socket 700, forming the petals 704 around the user's stump, and allowing the socket 700 to cool to a stabilized form. In an embodiment, a center 706 of the socket 700 connects the petals 704 and is used as location for reversibly affixing the socket 700 to the pole 124 of the prosthetic leg apparatus 100. Optionally, reversible affixing is accomplished using at least one hole 708 provided to the center 706. In some embodiments at least one upper cut 710 is provided to the socket 700 for passing at least one strap (not shown) therethrough. In some embodiments, at least one lower cut 712 is provided to the socket 700 to provide ventilation and/or for an additional strap for the user's stump inserted into the socket 700. In some aspects, apertures/holes are provided to the upper portion of socket 700 for aeration and/or for securing a strap therethrough. As with other embodiments described herein, gaps between petals of the socket 700 are optionally for facilitating freedom of movement and/or enhancing user comfort and/or easing socket self-fitting by the user.

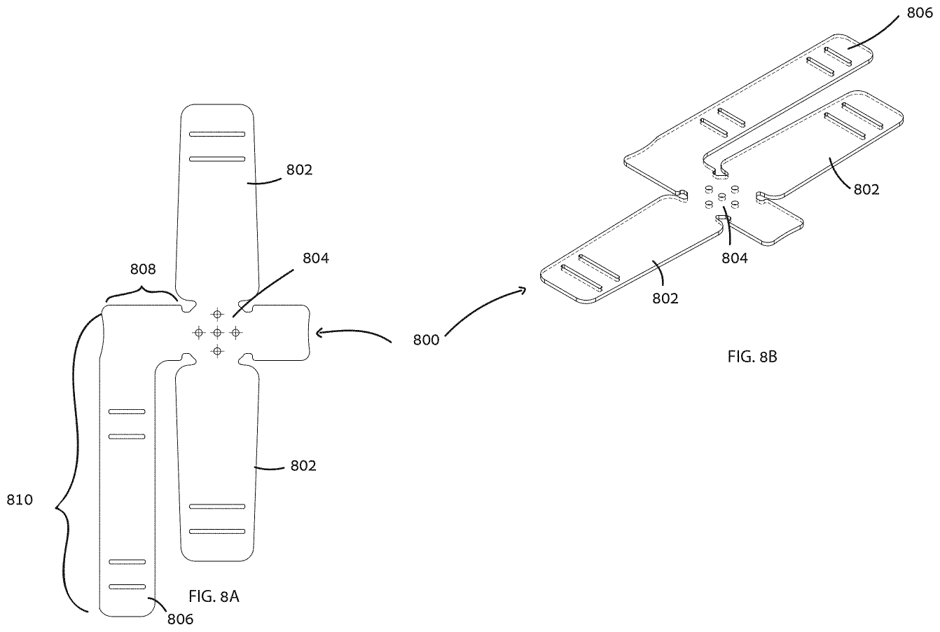

FIGS. 8A-12B are top and perspective views, respectively, of different sockets 800, 900, 1000, 1100, 1200 which each have different configurations but when formed are also commonly designed to provide structure which fully or partially encircles the user's stump. For example, socket 800 is provided with two symmetric petals 802 which, when formed around the user's stump, extend upwardly (towards the user's knee) from a center 804 which attaches to the pole 124, however an additional petal 806 is provided which is configured to fold upwards only a small increment 808 (relative to the overall height of petals 802) and which has an encircling portion which is of sufficient length 810 to wrap fully around the socket 800 when formed to the user's stump.

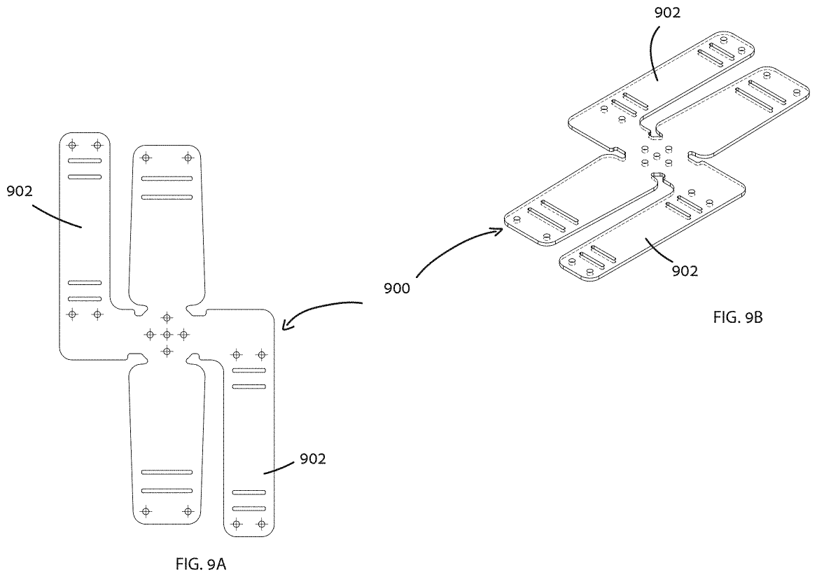

Socket 900 of FIGS. 9A-9B is similar to socket 800, but with two additional petals 902 for encirclement as opposed to just one.

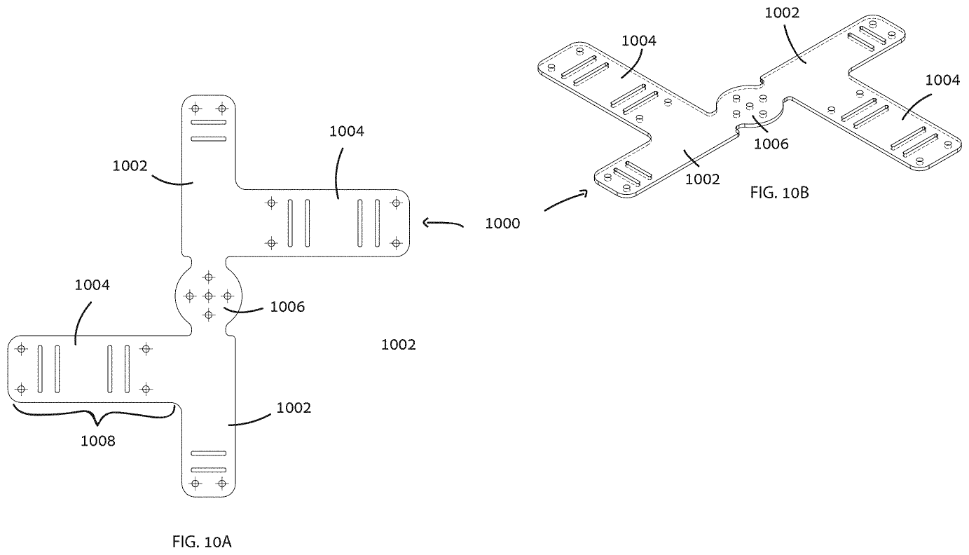

Socket 1000 of FIGS. 10A-10B shows a different configuration which is formed using a different methodology for achieving the encirclement result of the formed prosthetic leg apparatus 100. In an embodiment, socket 1000 is provided with petals 1002 which form upwardly from a center 1006, but which are also provided with side branch petals 1004, which when the petals 1002 are upwardly formed the side branch petals 1004 are in a position to permit encirclement of the user's stump. Optionally, each of the side branch petals 1004 covers an approximate semicircular portion of total encirclement, that is, both of the side branch petals 1004 in combination provide encirclement. In an embodiment, each side branch petal has a length 1008 which extends from one petal 1002 to the other petal 1002 when the socket 1000 is formed around the user's stump.

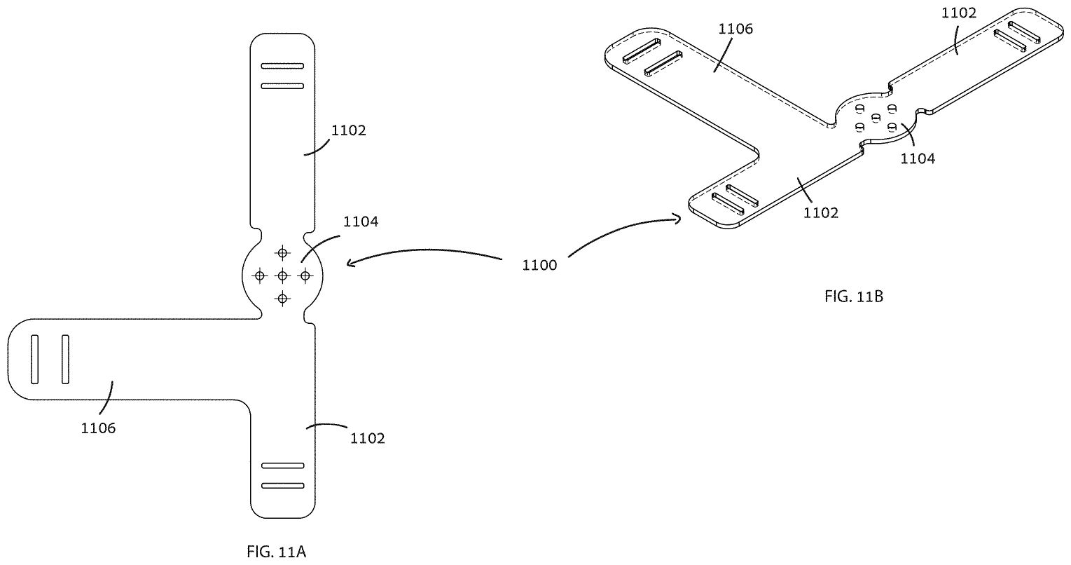

Socket 1100 of FIGS. 11A-11B shows a similar configuration to socket 1000, but with only one side branch petal 1106. Optionally, the side branch petal 1106 is of sufficient length to wrap all the way around petals 1102 when petals 1102 are upwardly formed from a center 1104, such that the socket 1100 is encircled when formed around the user's stump and side branch petal 1106 is wrapped around socket 1100.

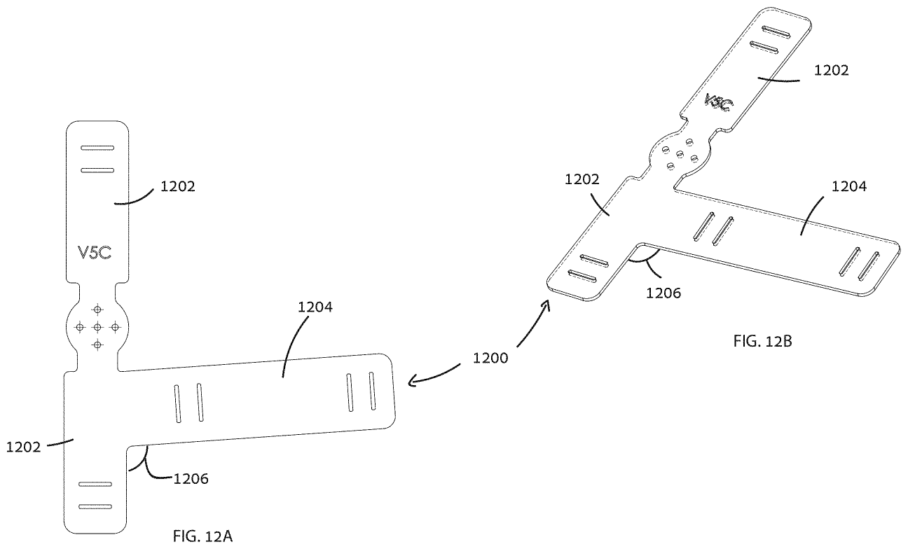

Socket 1200 of FIGS. 12A-12B is a configuration similar to socket 1100, however the side branch petal 1204 extends at an angle 1206 from the petal 1202 to better fit stump anatomy (which is often at least slightly asymmetric).

FIG. 15 is a perspective view of a formed socket 1500 with straps 1502, 1504, according to an embodiment. Strap 1502 is threaded through cuts 1506 made in the upper portion of the socket 1500 and is looped, where the loop would wrap around the leg of the user above the user's knee cap. In some embodiments, strap 1504 is threaded through at least one cut 1508 located in the lower portion of the socket 1500, wherein the cut is located under the user's knee cap.

FIG. 16 is a perspective view of a formed socket 1600 with a strap 1602, according to an embodiment. Strap 1602 is inserted into cuts 1604 made in the upper portion of the socket 1600 and is formed in a loop which would be wrapped around the user's leg. Cuts 1606 made into the lower portion of the socket 1600 are optionally usable for ventilation or additional, below the knee, straps.

FIGS. 17A-17B are top and bottom, respectively, perspective views of the base 122b of the adapter 122. FIG. 17A shows a plurality of holes 302, in accordance with an embodiment. FIG. 17B shows teeth 304, in accordance with an embodiment. Also shown in FIG. 18A is hole 306.

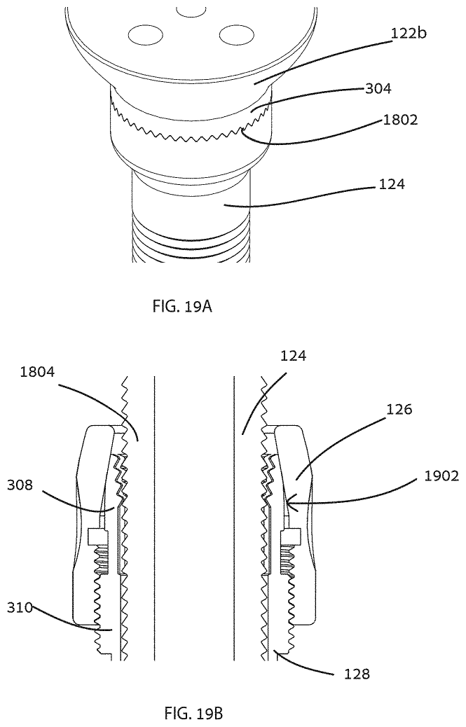

FIGS. 18A-18C are different views of the pole 124, in accordance with an embodiment. FIG. 18B shows a perspective view of an upper portion of the pole 124, showing in closer detail the teeth 1802 which are provided to the top of the pole to interface with the teeth 304 of the base 122b of the adapter 122 (shown in more detail in FIG. 19A), in an embodiment. FIG. 18C is a side, cross-sectional view of the pole 124, showing threading 1804 on one side of the pole 124 and smooth surface 1806 opposite the threading 1804.

FIG. 19A is a perspective view of the top of the pole 124 interfacing with the base 122b of the adapter 122, in an embodiment, where the teeth 304, 1802 mesh together to provide interconnection and/or to set the rotational orientation of the foot portion 150 with respect to the socket and by extension, the user's stump. FIG. 19B is a cross-sectional view of the bottom of the pole 124 inserted into the housing 128 and tightened with the nut 126. As described elsewhere herein, the taper 1902 of the nut 126 will urge petals 308 towards the threading 1804 of the pole 124 as the nut 126 is tightened onto the housing 128, urging the teeth of the petals into the threading 1804 to reversibly interlock the pole 124 to the housing 128 and setting a height of the pole 124. It should be understood that the nut 126 is selectively tightened when the pole height has reached a desirable level after height adjustment.

FIG. 23 is a perspective view of a socket 400 mounted on an adapter 122 of a pole 124.

FIG. 20A is a perspective view of the housing 128, in accordance with an embodiment. Shown in more detail are the petals 308, the threading 310 located on the outside of the top of the housing 128, the bottom 312 of the housing 128 and the at least one hole 318 for attaching the foot portion 150 to the housing 128 using a screw or a pin.

FIG. 20B is a cross-sectional view of the top of the housing 128 where the threading 310 and the petals 308 are located.

FIGS. 21A-21B are perspective and cross-sectional views, respectively, of the nut 126. FIG. 21B shows the tapered surface 1902 on the inside of the nut 126, in accordance with an embodiment. Additionally, the threaded inner surface 2102 of the nut 126 is shown in more detail. The threaded inner surface 2102 is designed as a counterpart to threading 310 located on the outside of the top of the housing 128.

FIGS. 22A-22B are perspective and side views, respectively, of the foot portion 150. The foot portion 150 design utilizes material properties along with shape and geometry, to achieve a combination of ergonomics, functionality, durability and performance. It is also conceived that the foot portion 150 can be offered in multiple interchangeable sizes (for example, for sequential use by a single user as he/she grows, or for different sized users) and/or is offered with different interchangeable configurations for different uses (for example, a foot portion configured for running and/or kicking a ball and/or is offered with various levels of traction and/or resiliency).

In some embodiments, the foot portion is designed to support the weight of a user during gait. Three stages of gait were tested during development: 1. Heel-strike—the initial stage of gait when an impact of 120% of body weight is exerted on the heel from the ground and the foot is at a 15 degrees angle from the ground; 2. Mid-stance—the middle stage of gait where the foot is horizontal and 100% of the body weight is exerted on the sole from the ground; and, 3. Heel-rise—the the final stage of gait when the foot is rolled at 20 degrees angle from the ground and 100% of the body weight is exerted at the front of the foot from the ground. The foot portion 150 is also designed to support loads which are exerted on the foot when it is misaligned, due to a misalignment with the socket and/or uneven surfaces on the ground. In some embodiments, at least part of the foot portion is constructed of polyoxymethylene.

In an embodiment, the foot portion 150 has a closed-loop main body shaped with a general outline of a human foot. In some embodiments, an outer wall thickness of the closed-loop main body is 4 mm-7 mm. Internal ribs 2202 are implemented for reinforcement, in some embodiments. In some embodiments, the internal ribs 2202 run from the top to the bottom of the main body and are approximately located at the nose (near the “toes”) and rear (near the heel). In some embodiments, the internal rib thickness is 3 mm-11 mm. Optionally, at least one internal rib 2202 is located near the middle of the main body. In an embodiment, the internal ribs 2202 prevent or reduce large deformation of the foot portion 150 during gait, while acting as a spring when bending. Enhanced structural strength and rigidity is optionally provided by at least one internal circumferential rib 2206.

In some embodiments, the sole 2204 has an at least slightly convex shape, which allows for a rolling motion of the foot portion 150, both for the direction of gait and the lateral direction, for stability on uneven/side sloped surfaces.

In some embodiments, the foot portion 150 has a rounded shape at the heel 2208. Positioning the radius of the heel 2208 at a longitudinal distance away from the anchor point (at the housing's 128 connection to the foot portion 150), contributes to the damping of reaction forces traveling up the pylon assembly 120 during heel strike. Much in the same manner, the sole 2204 is provided with a curve on the front 2210 such that spring tension could be stored during heel rise. This energy could then be released to assist with toe off.

In some embodiments, interconnection of the foot portion 150 and the housing 128 is simple and does not require special tools, for example using a click locking or snap-fit interface or by using a unified/integrated apparatus, such as described with respect to FIGS. 29-32D and elsewhere herein.

In some embodiments, the main body of the foot portion is approximately 150 mm-300 mm in length. In some embodiments, the main body of the foot portion is 60 mm -100 mm in width.

FIG. 29 is a perspective view of an ankle thread interface 2900, in accordance with an exemplary embodiment of the invention. In an embodiment, a top 2902 of the “ankle” of a prosthetic leg apparatus 2901 is configured with a plurality of serrated (e.g. threading facing towards the central axis of the apparatus 2901) petals 2904 formed in a ring where the central axis is the center of the ring) around the uppermost portion of the top 2902. In some embodiments, the petals 2904 taper (towards a central or long axis of the apparatus 100) and/or apply compressive pressure towards a lower part of a pylon 2906, which functions as at least a part of the “shin” of the user. At least the lower part of the pylon 2906 is configured with threading which is a counterpart to the threading in the inside surface of the petals 2904, such that the pylon 2906 can be screwed down and into the petals 2904.

In some embodiments of the invention, external threading 2908 is provided to the top 2902, for exemplary reasons described with respect to FIGS. 31A-31B, below.

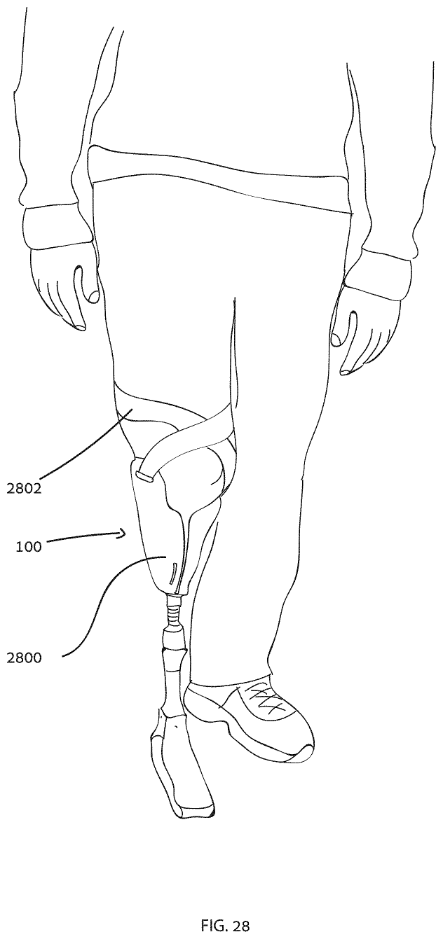

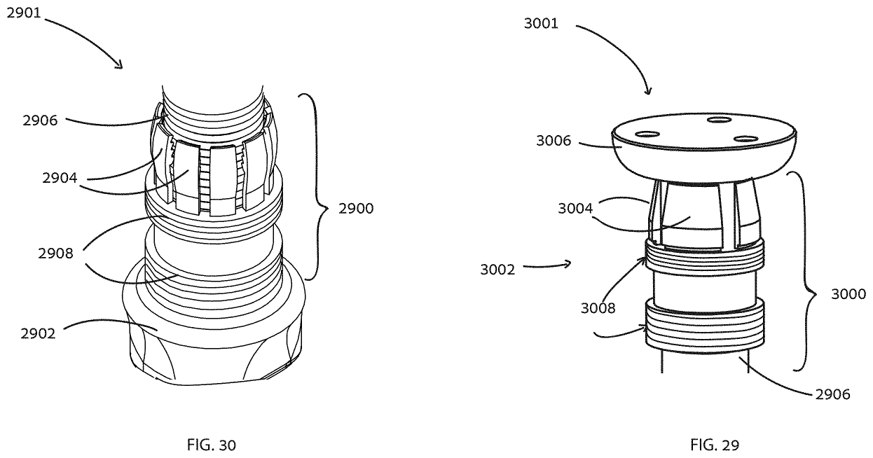

FIG. 30 is a perspective view of a pylon thread interface 3000, in accordance with an exemplary embodiment of the invention. In an embodiment, a top 3002 of the pylon 2906 is configured with a plurality of serrated (e.g. threading facing towards the central axis of the apparatus 3001) petals 3004 formed in a ring where the central axis is the center of the ring) around the uppermost portion of the top 3002. In some embodiments, the petals 3004 taper (towards a central or longitudinal axis of the apparatus 100) and/or apply compressive pressure towards an adapter 3006. In some embodiments, the adapter 3006 is provided with threading which is configured as a counterpart to the threading located on the inner facing surfaces of the petals 3004. In some embodiments, at least a portion of the external surface of the pylon 2906 is threaded for customizably attaching the pylon 2906 to the ankle thread interface 2900 shown in FIG. 29. In some embodiments of the invention, external threading 3008 is provided to the top of the pylon 2906, for exemplary reasons described with respect to FIGS. 31A-31B, below.

FIGS. 31A-31B are a perspective view and a cross-sectional view, respectively, of a substantially cylindrical nut 3100. As shown in FIG. 31B, the nut 3100 is provided with internal threading at location 3102 configured for use with the external threads 2908, 3008 of the ankle and pylon, in an embodiment of the invention. In some embodiments, the threading of the pylon and ankle are identical, so the same nut 3100 can be used for both. An internal taper 3104 is disposed near the top of the nut 3100, which due to the tapering applies pressure on the clamping petals 2904, 3004 of the pylon and/or ankle as the nut 3100 is screwed down wound the pylon and/or ankle which passes through the nut 3100 in direction 3106 (as the nut 3100 is being tightened).

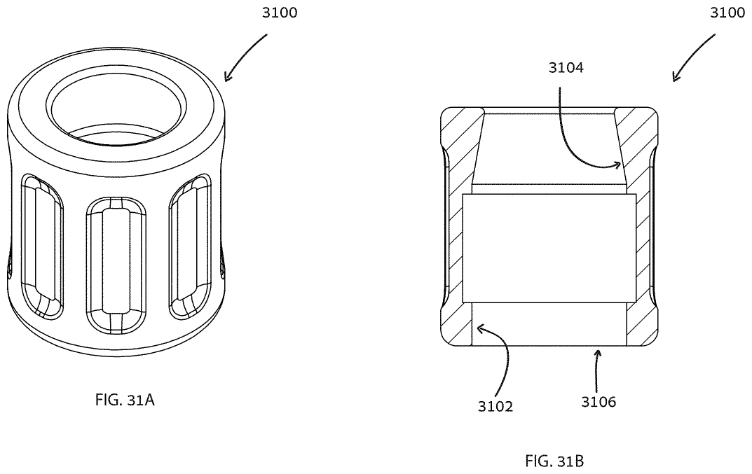

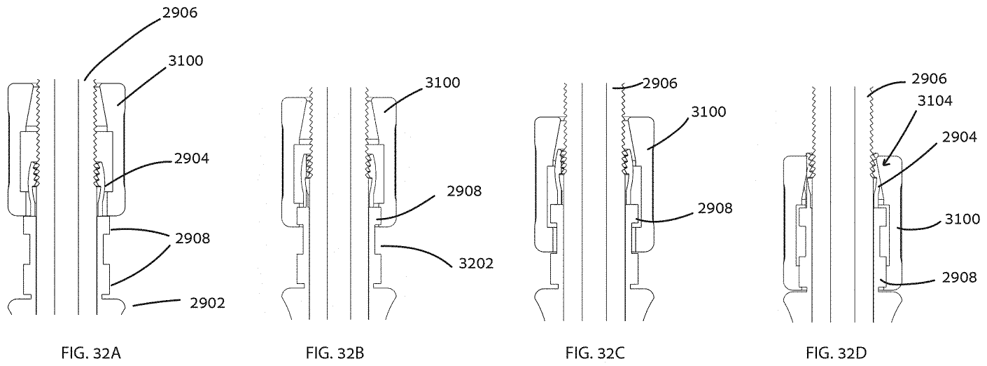

FIGS. 32A-32D are progressive cross-sectional views of a tool-less installation method. FIG. 32A shows the nut 3100 being placed around the pylon 2906 (or rather the pylon 2906 is placed through the nut 3100) and the nut 3100 is moved towards threading, for example ankle threading 2908. As shown in FIG. 32B, the nut 3100 first engages the top external threading 2908.

FIG. 32C shows the nut 3100 being screwed downwards until it disengages the upper-most external thread 2908. The internal threading 3102 of the nut 3100 sits fully inside a gap 3202 (the gap shown in more detail in FIGS. 29 and 30) between the external threads 2908 of the ankle thread interface 2900 and is typically removed from the interface by un-screwing it in the upward direction. In some embodiments, the nut 3100 is screwed past the upper threads 2908 and into the gap position shown in FIG. 32C, to allow for adjustment of the height of the pylon 2906 without the nut 3100 coming loose from the whole assembly.

FIG. 32D shows the nut 3100 screwed down such that the internal threading 3102 is engaged with the lower exteral threading 2908 while the internal tapering 3104 of the top portion of the nut 3100 is applying pressure to the petals 2904, which in turn apply compressive force to the pylon 2906 to help secure it in place. In an embodiment, the nut 3100 can be screwed back upwards until it disengages from the bottom external thread 2908 and positions the internal threading 3102 in the gap 3202 (such as shown in FIG. 32C).

FIG. 24 is a partially exploded view showing a bottom 312 of a housing 128 interfacing with a foot portion 150.

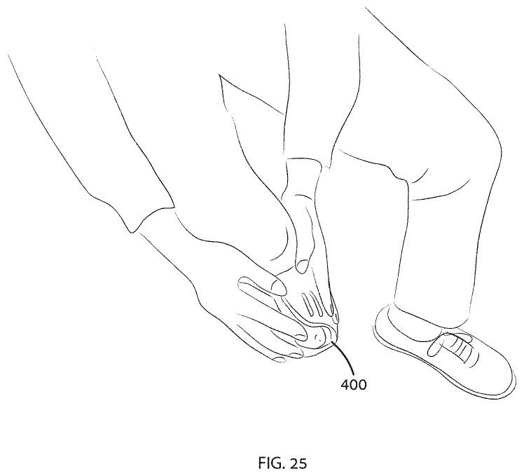

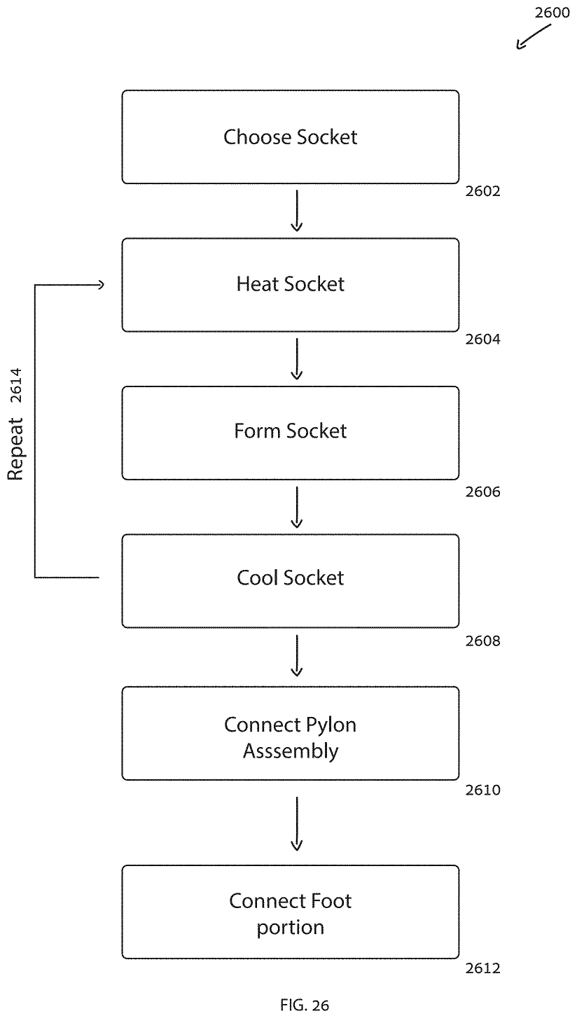

FIG. 26 is a flowchart 2600 of a method of using a prosthetic leg apparatus. In an embodiment, a socket is chosen (2602) for a particular user with particular needs. The socket is heated (2604) to a temperature sufficiently high to make the socket material malleable, formed (2606) to a stump of the user, and then allowed to cool (2608) while formed to the user's stump such that the cooled socket maintains the shape of the stump. The socket is then optionally reversibly connected (2610) to a pylon assembly, which is reversibly pre-connected or is subsequently reversibly connected (2612) to a foot portion. Optionally, the heating (2604), forming (2606) and cooling (2608) is repeated (2614) for subsequent customization of the socket to the user.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range. Further, described ranges are intended to include numbers outside any range described within statistical error and/or inherent measurement equipment limitations.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

1. A prosthetic leg apparatus adaptable for a user's stump, comprising:
a socket, wherein the socket is constructed of a thermoplastic material;
a pylon assembly having a longitudinal axis, wherein the pylon assembly extends within the longitudinal axis, connected to the socket and adjustable in length; and,
a foot portion, connected to the pylon assembly.

2-3. (canceled)

4. A prosthetic leg apparatus according to claim 1, wherein the pylon assembly includes at least one of an adapter, a pole, a nut and a housing.

5. A prosthetic leg apparatus of claim 4, wherein the adapter comprises a cap and a base.

6. A prosthetic leg apparatus of claim 4, wherein the base is provided with serrations or teeth which are formed as counterparts to serrations or teeth located at a top of the pole.

7. A prosthetic leg apparatus according to claim 4, wherein the pole is circular in shape at a top of the pole and D-shaped at a bottom of the pole.

8. A prosthetic leg apparatus according to claim 4, wherein the housing is provided with a plurality of petals at a top of the housing.

9. A prosthetic leg apparatus of claim 8, wherein an exterior surface of the pole is threaded to match inner facing, threaded surfaces of the plurality of petals.

10. A prosthetic leg apparatus according to claim 4, wherein the nut is cylindrical in shape and is disposed around the pole, where the pole passes through a center of the nut.

11. A prosthetic leg apparatus of claim 10, wherein the nut is configured with a threaded inner surface.

12. A prosthetic leg apparatus of claim 11, wherein a top of the housing is provided with threading which is configured as a counterpart to the threaded inner surface.

13. (canceled)

14. A prosthetic leg apparatus of claim 7, wherein at least a portion of the housing is D-shaped on the inside to match the D-shape of the pole, such that the pole is slidable within the housing but not rotatable around a longitudinal axis of the pylon assembly.

15. A prosthetic leg apparatus according to claim 1, wherein the foot portion includes at least one internal rib.

16. (canceled)

17. A prosthetic leg apparatus according to claim 1, further comprising a cover attached to the pylon assembly configured with an anatomically correct shape of a lower leg.

18. A method of using a prosthetic leg apparatus with a user's stump, comprising:
choosing a socket for the user's stump;
heating the socket to a temperature sufficiently high to make the socket malleable;
forming the socket around the user's stump;
cooling the socket to a temperature where the socket is rigid; and,
connecting at least a pylon assembly, having a longitudinal axis, wherein the pylon assembly extends within the longitudinal axis, to the socket.

19. A method of claim 18, further comprising connecting a foot portion to the pylon assembly.

20. A method according to claim 18, further comprising repeating the heating, forming and cooling to re-configure the socket to the user's stump.

21-36. (canceled)

37. A foot portion of a prosthetic leg assembly, comprising:
a closed-loop main body shaped with a general outline of a human foot; and,
an opening provided on the top of the main body configured as a counterpart to a bottom of a housing of a pylon assembly of the prosthetic leg assembly.

38. A foot portion according to claim 37, further comprising at least one internal rib extending from a top of the main body to a bottom of the main body.

39. A foot portion according to claim 37, further comprising at least one circumferential rib extending around an inner surface of the main body.

40-46. (canceled)

47. A foot portion according to claim 37, provided with a tool-less interface between the opening and the housing.

48. (canceled)
